(19) United States
(12) Patent Application Publication
(10) Pub. No.: US 2024/0084298 A1
(43) Pub. Date: Mar. 14, 2024

(54) Treatment Of Endometriosis With R-Spondin 3 (RSPO3) Inhibitors

(71) Applicant: Regeneron Pharmaceuticals, Inc., Tarrytown, NY (US)

(72) Inventors: Manuel Allen Revez Ferreira, Tarrytown, NY (US); Maria Cristina Suciu, Tarrytown, NY (US); Courtney Williams, Tarrytown, NY (US)

(21) Appl. No.: 18/244,577

(22) Filed: Sep. 11, 2023

Related U.S. Application Data
(60) Provisional application No. 63/405,787, filed on Sep. 12, 2022.
(60) Provisional application No. 63/414,748, filed on Oct. 10, 2022.

(51) Int. Cl. C12N 15/113; A61K 45/06; C07K 16/18

(52) U.S. Cl. CPC C12N 15/113; A61K 45/06; C07K 16/18; A61K 2039/505

(57) ABSTRACT

The present disclosure relates generally to the treatment of subjects having endometriosis or at risk of developing endometriosis by administering an R-spondin 3 (RSPO3) inhibitor and/or an RSPO3 Receptor blocker to the subject.

### FIELD

The present disclosure generally relates to the treatment of subjects having endometriosis or who are at risk of developing endometriosis by administering an R-Spondin 3 (RSPO3) inhibitor or an RSPO3 Receptor blocker, and to methods of identifying subjects having an increased risk of developing endometriosis.

### BACKGROUND

Endometriosis is a disease of adolescents and reproductive-aged woman that is defined as the presence of endometrial tissue outside of the uterine cavity. Endometriosis affects 10-15% of women of reproductive age globally and often results in chronic pelvic pain and infertility. Despite its prevalence, for many women there is often a significant delay in diagnosing endometriosis and misdiagnosis is common, resulting in years of unnecessary suffering and a reduction in quality of life. Moreover, while several treatments are available for treating endometriosis, such treatments may not be a permanent “fix”, since 20%-40% of women experience a return of symptoms following cessation of treatment. Thus, there is a need for earlier and more accurate methods of diagnosing endometriosis, and for treating the disease once it has been diagnosed.

Roof plate-specific spondin (RSPO) genes are a family of genes that are important regulators of the WNT signaling pathway, which is an ancient and evolutionarily conserved pathway involved in regulation of cell fate determination, cell migration, cell polarity, and organogenesis during embryonic development. In mammals, the RSPO family consists of four members, RSPO1 through RSPO4, which encode proteins sharing between 40%-60% amino acid sequence identity, and substantial structural homology. Studies using these proteins have identified multiple RSPO receptors that are involved in regulation of the WNT signaling pathway. The RSPO3 protein, also known as Pwtsr and Thsd2, is a 272 amino acid protein that acts as a ligand for the LGR4-6 receptor, a key regulator of angiogenesis, and that is encoded by the RSPO3 gene, which spans nucleotides 127,118,671-127,199,481 on human chromosome 6 (GRCh38:CM000668.2). The gene has three transcripts (i.e., splice variants) and 242 orthologues have been identified to date. So far, over 100 single nucleotide polymorphisms (SNPs) have been mapped to the RSPO3 gene, and the gene has been linked to at least 62 phenotypes. However, to date, no RSPO3 SNPs have been associated with endometriosis.

### SUMMARY

The present disclosure provides methods of treating a subject having endometriosis, or at risk of developing endometriosis, the methods comprising administering an RSPO3 inhibitor and/or an RSPO3 Receptor blocker to the subject.

The present disclosure also provides methods of treating a subject having endometriosis or at risk of developing endometriosis by administering an endometriosis therapeutic agent or an endometriosis therapy, the methods comprising: determining or having determined whether the subject has an RSPO3 variant nucleic acid molecule, by: obtaining or having obtained a biological sample from the subject; and performing or having performed a sequence analysis on the biological sample to determine if the subject has a genotype comprising an RSPO3 variant nucleic acid molecule; and administering or continuing to administer the endometriosis therapeutic agent or endometriosis therapy in a standard dosage amount to a subject that is RSPO3 reference; or administering or continuing to administer the endometriosis therapeutic agent in an amount that is the same as or less than a standard dosage amount or endometriosis therapy to a subject that is heterozygous or homozygous for the RSPO3 variant nucleic acid molecule, and/or administering an RSPO3 inhibitor and/or an RSPO3 Receptor blocker to the subject; wherein the presence of a genotype having the RSPO3 variant nucleic acid molecule indicates the subject has an increased risk of developing endometriosis.

The present disclosure also provides methods of identifying a subject having an increased risk of developing endometriosis, the methods comprising: determining or having determined the presence or absence of an RSPO3 variant nucleic acid molecule in a biological sample obtained from the subject; wherein: when the subject is RSPO3 reference, then the subject has a decreased risk of developing endometriosis; and when the subject is heterozygous or homozygous for the RSPO3 variant nucleic acid molecule, then the subject has an increased risk of developing endometriosis.

The present disclosure also provides endometriosis therapeutic agents for use in the treatment or prevention of endometriosis in a subject having an RSPO3 variant nucleic acid molecule.

The present disclosure also provides RSPO3 inhibitors and/or RSPO3 Receptor blockers for use in the treatment or prevention of endometriosis in a subject that is heterozygous or homozygous for an RSPO3 variant nucleic acid molecule.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows summary statistics results for the lead variant (r59482771, 6:127125465:G:C) at RSPO3 locus associated with endometriosis, in each of the nine studies contributing to the endometriosis meta-analysis, as well as for the meta-analysis.

FIG. 2 shows a comparison of effect sizes in endometriosis and in the RSPO3 protein level study, at variants in RSPO3 locus. The coloured lines represent the estimates from three statistical tests in mendelian randomization (MR) analysis which show that lower effect on the protein is associated with lower effect on the disease. Circles in the plot represent both variants nearby protein-cis (filled dots), as well as variants which impact the protein from other genomic locations-trans (hollow dots). The second part of FIG. 2 shows the statistics associated with the mendelian randomization tests, where cis inverse variance weighted methods (cis-IVW) si most strongly associated. The third part of FIG. 2 illustrates the MR analysis estimates for the method most associated, cis-IVW, in all the internal cohorts contributing to the meta-analysis.

FIG. 3 shows that the endometriosis lead variant and the protein levels variant (pQTL) underlie the same genetic signal. Shown are regional association plots at RSPO3, in endometriosis (above) and protein levels (below), for the lead variant for endometriosis (r59482771, 6:127125465:G:C) and lead variant for the protein levels (r51936800, 6:127114919:C:T). These variants are in high linkage disequilibrium (LD) in European population (r2=0.88) and are co-inherited, confirming colocalization of the two signals.

FIG. 4 shows RSPO3 expression and other WNT signaling genes; RSPO3 expression was mostly observed on smooth muscles and fibroblasts and some expression on endothelial cells.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternately phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horses, cows, and pigs), companion animals (such as, for example, dogs and cats), laboratory animals (such as, for example, mice, rats, and rabbits), and non-human primates. In some embodiments, the subject is a human. In some embodiments, the human is a patient under the care of a physician.

It has been observed in accordance with the present disclosure that RSPO3 variant nucleic acid molecules (whether these variants are homozygous or heterozygous in a particular subject) associate with an increased risk of developing endometriosis. Although RSPO3 has been associated with fibrosis, it is believed that RSPO3 variant nucleic acid molecules have not been clearly associated with endometriosis in humans. Therefore, subjects that are heterozygous or homozygous for an RSPO3 variant nucleic acid molecule may be treated with an RSPO3 inhibitor and/or an RSPO3 Receptor blocker such that endometriosis is inhibited or prevented, the symptoms thereof are reduced or prevented, and/or development of symptoms is repressed or prevented. It is also believed that such subjects having endometriosis may further be treated with one or more endometriosis therapeutic agents or endometriosis therapy that treats or inhibits endometriosis. In addition, the present disclosure provides methods of leveraging the presence or absence of RSPO3 variant nucleic acid molecules in subjects to identify or stratify risk is such subjects of developing endometriosis, or to diagnose subjects as having an increased risk of developing endometriosis.

For purposes of the present disclosure, any particular subject, such as a human, can be categorized as having one of three RSPO3 genotypes: i) RSPO3 reference; ii) heterozygous for an RSPO3 variant nucleic acid molecule; or iii) homozygous for an RSPO3 variant nucleic acid molecule. A subject is RSPO3 reference when the subject does not have a copy of an RSPO3 variant nucleic acid molecule. A subject is heterozygous for an RSPO3 variant nucleic acid molecule when the subject has a single copy of an RSPO3 variant nucleic acid molecule. A subject is homozygous for an RSPO3 variant nucleic acid molecule when the subject has two copies of an RSPO3 variant nucleic acid molecule.

RSPO3 variant nucleic acid molecules can be any nucleic acid molecule (such as, a genomic nucleic acid molecule, an mRNA molecule, or a cDNA molecule produced from an mRNA molecule) that results in the increased expression or activity of an RSPO3 polypeptide. In some embodiments, the RSPO3 variant nucleic acid molecule is a missense variant nucleic acid molecule. In some embodiments, the RSPO3 variant nucleic acid molecule comprises a variation in a coding region. In some embodiments, the RSPO3 variant nucleic acid molecule icomprises a variation that is cis in regard to the RSPO3 gene. In some embodiments, the RSPO3 variant nucleic acid molecule icomprises a variation that is trans in regard to the RSPO3 gene. In some embodiments, the RSPO3 variant nucleic acid molecule comprises a variation in a non-coding region. In some embodiments, the RSPO3 variant nucleic acid molecule does not comprise a variation in a non-coding region, except for splice acceptor regions (two bases before the start of any exon except the first). In some embodiments, the RSPO3 variant nucleic acid molecule is a splice-site variant, a stop-gain variant, a start-loss variant, a stop-loss variant, a frameshift variant, or an in-frame indel variant. In some embodiments, the RSPO3 variant nucleic acid molecule is any nucleic acid molecule resulting in increased expression of RSPO3 mRNA or RSPO3 polypeptide. In some embodiments, the RSPO3 variant nucleic acid molecule is a variant that causes or is predicted to cause a nonsynonymous amino-acid substitution in a nucleic acid molecule and whose allele frequency is less than 1/100 alleles in the population from which the subject is selected. In some embodiments, the RSPO3 variant nucleic acid molecule is any rare missense variant (allele frequency <0.1%; or 1 in 1,000 alleles), or any splice-site, stop-gain, start-loss, stop-loss, frameshift, or in-frame indel, or other frameshift RSPO3 variant.

In any of the embodiments described herein, the RSPO3 variant genomic nucleic acid molecule can include one or more variations at any of the positions of chromosome 6 (i.e., positions 127,118,671-127,199,481) using the nucleotide sequence of the RSPO3 reference genomic nucleic acid molecule in the GRCh38/hg38 human genome assembly (see, ENSG00000146374 annotated in the in the Ensembl database (URL: world wide web at “uswest.ensembl.org/Homo_sapiens/Gene/”)) as a reference sequence. The sequences provided in these transcripts for the RSPO3 genomic nucleic acid molecule are only exemplary sequences. Other sequences for the RSPO3 genomic nucleic acid molecule are also possible.

In any of the embodiments described herein, the RSPO3 variant nucleic acid molecule can comprise any one or more of the following genetic variations (referring to the chromosome:positions set forth in the GRCh38/hg38 human genome assembly): 6:127125465:G:C (r59482771), 6:126727085:C:T (r5111743285), 6:126973497:T:C (r59398828), 6:127017612:T:C (r5117698897), 6:127114919:C:T (r51936800), 6:127118748:C:G (r5138650988), 6:127118902:T:C (r5577721086), 1:19979653:G:C (r511573156), 3:56815721:T:C (r51354034), 3:136318144:T:C (r5664088), 6:2507158:T:C (r5879097), 6:117184879:T:G (r5200564310), 6:154441670:T:C (r575045626), 6:160671406:C:T (r511751347), 6:160706469:A:G (r573015965), 6:160716958:G:A (r5783147), 6:160731873:C:T (r54252129), 6:160741337:T:A (r5147175166), 7:151716108:T:C (r510224210), 8:105578477:C:A (r54541868), 12:71474678:T:C (r560195346), 13:73551661:G:A (r51887646), 14:54766806:G:A (r57147275), 14:54768564:C:T (r54901541), 14:94371805:G:T (r5112635299), 16:251942:A:G (rs9934955), 16:3697041:G:T (r51635404), 17:28367840:G:A (rs704), 19:44846145:T:C (r53810143), 19:55027612:T:C (r51654425), 20:45347027:A:C (r51981430), 20:45354752:G:A (r56065808), and 23:70475947:G:T. In any of the embodiments described herein, the RSPO3 variant nucleic acid molecule can comprise any one or more of the following genetic variations: 6:127125465:G:C (rs9482771), 6:126727085:C:T (rs111743285), 6:126973497:T:C (rs9398828), 6:127017612:T:C (rs117698897), 6:127114919:C:T (rs1936800), 6:127118748:C:G (rs138650988), and 6:127118902:T:C (rs577721086).

In any of the embodiments described herein, the RSPO3 variant nucleic acid molecule can comprise the genetic variation 6:127125465:G:C (r59482771). In some embodiments, the RSPO3 variant nucleic acid molecule is a RSPO3 variant genomic nucleic acid molecule that comprises the genetic variant rs9482771, or is an mRNA molecule produced therefrom, or is a cDNA molecule produced from the mRNA molecule. In some embodiments, the RSPO3 variant nucleic acid molecule is a RSPO3 variant genomic nucleic acid molecule that comprises a cytosine at position 127,125,465 of chromosome 6, or is an mRNA molecule produced therefrom, or is a cDNA molecule produced from the mRNA molecule.

For subjects that are genotyped or determined to be heterozygous or homozygous for an RSPO3 variant nucleic acid molecule, such subjects have an increased risk of developing endometriosis. For subjects that are genotyped or determined to be heterozygous or homozygous for an RSPO3 variant nucleic acid molecule, such subjects can be treated with an RSPO3 inhibitor and/or an RSPO3 Receptor blocker.

In any of the embodiments described herein, the subject in whom endometriosis is prevented by administering an RSPO3 inhibitor and/or an RSPO3 Receptor blocker can be anyone at risk for developing endometriosis including, but not limited to, subjects with a genetic predisposition for developing endometriosis. Additional risk factors for endometriosis may include, but are not limited to, having a family member diagnosed with endometriosis, being nulliparous, starting menstruation at an early age (e.g., before 11 years of age), short menstrual cycles (e.g., less than 27 days), heavy menstrual cycles lasting greater than seven days, and going through menopause at an older age. In some embodiments, administering an RSPO3 inhibitor and/or an RSPO3 Receptor blocker to a subject having endometriosis may be carried out to prevent development of another of endometriosis event in a subject who has already had endometriosis. In any of the embodiments described herein, the methods can be used to improve endometriosis.

Any one or more (i.e., any combination) of the RSPO3 variant nucleic acid molecules described herein can be used within any of the methods described herein to determine whether a subject has an increased risk of developing endometriosis. The combinations of particular variants can form a mask used for statistical analysis of the particular correlation of RSPO3 and an increased risk of developing endometriosis. In some embodiments, the mask used for statistical analysis of the particular correlation of RSPO3 and an increased risk of developing endometriosis can exclude any one or more of these RSPO3 variant nucleic acid molecules described herein.

In any of the embodiments described herein, the subject can have endometriosis. In any of the embodiments described herein, the subject can be at risk of developing endometriosis. In any of the embodiments described herein, the endometriosis may comprise superficial peritoneal endometriosis, endometrioma-containing endometriosis, deeply infiltrating endometriosis (DIE), or abdominal wall endometriosis. In some embodiments, the endometriosis may be superficial peritoneal endometriosis. In some embodiments, the endometriosis may be endometrioma-containing endometriosis. In some embodiments, the endometriosis may be DIE. In some embodiments, the endometriosis may be abdominal wall endometriosis.

In any of the embodiments described herein, the methods can be used to treat a complication or co-morbidity of endometriosis, or reduce the risk of developing the same. Complications of endometriosis may include, but are not limited to, dysmenorrhea, pain upon intercourse, impaired fertility, adhesions, ovarian cysts, bladder problems, bowel problems, fatigue, diarrhea, constipation, bloating, and nausea. Co-morbidities of endometriosis may include, but are not limited to, ovarian cancer, breast cancer, melanoma, immune disorders (e.g., systemic lupus erythematosus, Sjogren's syndrome, multiple sclerosis (MS), rheumatoid arthritis (RA), inflammatory bowel diseases (IBD) such as Chrohn's disease, ulcerative colitis (UC), and coeliac disease (CD)), allergic diseases (e.g., eczema, hay fever, asthma and sensitivities to food or medications), and cardiovascular diseases (e.g., myocardial infarction and angina).

The present disclosure provides methods of treating a subject having endometriosis or at risk of developing endometriosis, the methods comprising administering an RSPO3 inhibitor and/or an RSPO3 Receptor blocker to the subject.

In some embodiments, the RSPO3 inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of an RSPO3 nucleic acid molecule. In some embodiments, the antisense RNA, siRNA, or shRNA hybridizes to a sequence within an RSPO3 genomic nucleic acid molecule or mRNA molecule and decreases expression of the RSPO3 polypeptide in a cell in the subject. In some embodiments, the RSPO3 inhibitor comprises an antisense molecule that hybridizes to an RSPO3 genomic nucleic acid molecule or mRNA molecule and decreases expression of the RSPO3 polypeptide in a cell in the subject. In some embodiments, the RSPO3 inhibitor comprises an siRNA that hybridizes to an RSPO3 genomic nucleic acid molecule or mRNA molecule and decreases expression of the RSPO3 polypeptide in a cell in the subject. In some embodiments, the RSPO3 inhibitor comprises an shRNA that hybridizes to an RSPO3 genomic nucleic acid molecule or mRNA molecule and decreases expression of the RSPO3 polypeptide in a cell in the subject.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀ alkenyl, and C₂₋₁₀ alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, CI, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH 2 and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and anninoalkylphosphorannidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

Sense: mN*mN*/i2FN/mN/i2FN/mN/i2FN/mN/i2FN/mN/i2FN/mN/i2FN/mN/i2FN/mN/i2FN/*mN*/32FN/

Antisense: /52FN/*/i2FN/*mN/i2FN/mN/i2FN/mN/i2FN/mN/i2FN/mN/i2FN/mN/i2FN/mN/i2FN/mN/i2FN/mN*N*N

wherein: “N” is the base; “2F” is a 2′-F modification; “m” is a 2′-O-methyl modification, “I” is an internal base; and “*” is a phosphorothioate backbone linkage.

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the RSPO3 inhibitor comprises a nuclease agent that induces one or more nicks or double-strand breaks at a recognition sequence(s) or a DNA-binding protein that binds to a recognition sequence within an Rspo3 genomic nucleic acid molecule. The recognition sequence can be located within a coding region of the RSPO3 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the RSPO3 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify an RSPO3 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of RSPO3 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in an RSPO3 genomic nucleic acid molecule or it can be a nickase that creates a single-strand break in an RSPO3 genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (Cas6), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. In some embodiments, a Cas system, such as Cas12a, can have multiple gRNAs encoded into a single crRNA. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of RSPO3 genomic nucleic acid molecules can be generated by contacting a cell with a Cas protein and one or more gRNAs that hybridize to one or more gRNA recognition sequences within a target genomic locus in the RSPO3 genomic nucleic acid molecule. The gRNA recognition sequence can include or be proximate to the start codon of an RSPO3 genomic nucleic acid molecule or the stop codon of an RSPO3 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in an RSPO3 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within an RSPO3 genomic nucleic acid molecule. An exemplary gRNA is a gRNA effective to direct a Cas enzyme to bind to or cleave an RSPO3 genomic nucleic acid molecule, wherein the gRNA comprises a DNA-targeting segment that hybridizes to a gRNA recognition sequence within the RSPO3 genomic nucleic acid molecule. Exemplary gRNAs comprise a DNA-targeting segment that hybridizes to a gRNA recognition sequence present within an RSPO3 genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

The Cas protein and the gRNA form a complex, and the Cas protein cleaves the RSPO3 genomic nucleic acid molecule. The Cas protein can cleave the nucleic acid molecule at a site within or outside of the nucleic acid sequence present in the RSPO3 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the RSPO3 genomic nucleic acid molecule to which a DNA-targeting segment of a gRNA will bind.

Such methods can result, for example, in an RSPO3 genomic nucleic acid molecule in which a region of the RSPO3 genomic nucleic acid molecule is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the Rspo3 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In any of the methods of treatment or prevention described herein, the subject being treated can comprise an RSPO3 variant nucleic acid molecule. In some embodiments, the subject being treated is heterozygous for the RSPO3 variant nucleic acid molecule. In some embodiments, the subject being treated is homozygous for the RSPO3 variant nucleic acid molecule. The RSPO3 variant nucleic acid molecule can be any of the RSPO3 variant nucleic acid molecules disclosed herein. In some embodiments, the RSPO3 variant nucleic acid molecule is a RSPO3 variant genomic nucleic acid molecule that comprises the genetic variation of rs9482771, or is an mRNA molecule produced therefrom, or is a cDNA molecule produced from the mRNA molecule.

In some embodiments, the methods of treatment or prevention further comprise detecting the presence or absence of an RSPO3 variant nucleic acid molecule in a biological sample from the subject. In some embodiments, the RSPO3 variant nucleic acid molecule can be any of the RSPO3 variant nucleic acid molecules disclosed herein. In some embodiments, the RSPO3 variant nucleic acid molecule comprises any one or more of the following genetic variations: 6:127125465:G:C (r59482771), 6:126727085:C:T (r5111743285), 6:126973497:T:C (r59398828), 6:127017612:T:C (r5117698897), 6:127114919:C:T (r51936800), 6:127118748:C:G (r5138650988), 6:127118902:T:C (r5577721086), 1:19979653:G:C (r511573156), 3:56815721:T:C (r51354034), 3:136318144:T:C (r5664088), 6:2507158:T:C (r5879097), 6:117184879:T:G (r5200564310), 6:154441670:T:C (r575045626), 6:160671406:C:T (r511751347), 6:160706469:A:G (r573015965), 6:160716958:G:A (r5783147), 6:160731873:C:T (r54252129), 6:160741337:T:A (r5147175166), 7:151716108:T:C (r510224210), 8:105578477:C:A (r54541868), 12:71474678:T:C (r560195346), 13:73551661:G:A (r51887646), 14:54766806:G:A (r57147275), 14:54768564:C:T (r54901541), 14:94371805:G:T (r5112635299), 16:251942:A:G (rs9934955), 16:3697041:G:T (r51635404), 17:28367840:G:A (rs704), 19:44846145:T:C (r53810143), 19:55027612:T:C (r51654425), 20:45347027:A:C (r51981430), 20:45354752:G:A (r56065808), and 23:70475947:G:T. In some embodiments, the RSPO3 variant nucleic acid molecule comprises any one or more of the following genetic variations: 6:127125465:G:C (rs9482771), 6:126727085:C:T (rs111743285), 6:126973497:T:C (rs9398828), 6:127017612:T:C (rs117698897), 6:127114919:C:T (rs1936800), 6:127118748:C:G (rs138650988), and 6:127118902:T:C (rs577721086). In some embodiments, the RSPO3 variant nucleic acid molecule comprises the genetic variation 6:127125465:G:C (r59482771).

The present disclosure also provides methods of treating a subject with an endometriosis therapeutic agent or endometriosis therapy that treats or inhibits endometriosis, wherein the subject has endometriosis or is at risk of developing endometriosis. The methods comprise determining whether the subject has an RSPO3 variant nucleic acid molecule by obtaining or having obtained a biological sample from the subject and performing or having performed a sequence analysis on the biological sample to determine if the subject has a genotype comprising the RSPO3 variant nucleic acid molecule. In embodiments where the subject is RSPO3 reference, the methods further comprise administering or continuing to administer the endometriosis therapeutic agent in a standard dosage amount or endometriosis therapy. In embodiments where the subject is heterozygous or homozygous for the RSPO3 variant nucleic acid molecule, the methods further comprise administering or continuing to administer the endometriosis therapeutic agent in an amount that is the same as or less than a standard dosage amount or endometriosis therapy, and/or administering an RSPO3 inhibitor and/or an RSPO3 Receptor blocker to the subject. The presence of a genotype having the RSPO3 variant nucleic acid molecule indicates the subject has an increased risk of developing endometriosis. In some embodiments, the subject is RSPO3 reference. In some embodiments, the subject is heterozygous for an RSPO3 variant nucleic acid molecule. In some embodiments, the subject is homozygous for an RSPO3 variant nucleic acid molecule. In any of the embodiments described herein, the RSPO3 inhibitor and/or an RSPO3 Receptor blocker is an example of an endometriosis therapeutic agent. In some embodiments, the RSPO3 variant nucleic acid molecule is an RSPO3 variant genomic nucleic acid molecule that comprises the genetic variation rs9482771, or is an mRNA molecule produced therefrom, or is a cDNA molecule produced from the mRNA molecule.

For subjects that are genotyped or determined to be heterozygous or homozygous for an RSPO3 variant nucleic acid molecule, such subjects can be administered an RSPO3 inhibitor and/or an RSPO3 Receptor blocker, as described herein.

Detecting the presence or absence of an RSPO3 variant nucleic acid molecule in a biological sample from a subject and/or determining whether a subject has an RSPO3 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when the subject is RSPO3 reference, the subject is administered an endometriosis therapeutic agent in a standard dosage amount or endometriosis therapy that treats, prevents, or inhibits endometriosis. In some embodiments, when the subject is heterozygous or homozygous for an RSPO3 variant nucleic acid molecule, the subject is administered an endometriosis therapeutic agent in a dosage amount that is the same as or less than a standard dosage amount or endometriosis therapy that treats, prevents, or inhibits endometriosis, and/or an RSPO3 inhibitor and/or an RSPO3 Receptor blocker.

In some embodiments, the treatment or prevention methods comprise detecting an increase in the expression of RSPO3 mRNA or protein in a biological sample from the subject. In some embodiments, when the subject does not have an increase in the expression of RSPO3 mRNA or protein, the subject is administered an endometriosis therapeutic agent in a standard dosage amount or endometriosis therapy that treats, prevents, or inhibits endometriosis. In some embodiments, when the subject has an increase in the expression of RSPO3 mRNA or protein, the subject is administered an endometriosis therapeutic agent in a dosage amount that is the same as or less than a standard dosage amount or endometriosis therapy that treats, prevents, or inhibits endometriosis, and/or an RSPO3 inhibitor and/or an RSPO3 Receptor blocker.

The present disclosure also provides methods of treating a subject with an endometriosis therapeutic agent or endometriosis therapy that treats or inhibits endometriosis, wherein the subject has endometriosis or is at risk of developing endometriosis. The methods comprise determining whether the subject has an increase in the expression of RSPO3 mRNA or protein by obtaining or having obtained a biological sample from the subject and performing or having performed an assay on the biological sample to determine if the subject has an increase in the expression of RSPO3 mRNA or protein. When the subject does not have an increase in the expression of RSPO3 mRNA or protein, the endometriosis therapeutic agent in a standard dosage amount or endometriosis therapy is administered or continued to be administered to the subject. When the subject has an increase in the expression of RSPO3 mRNA or protein, the endometriosis therapeutic agent in an amount that is the same as or less than a standard dosage amount or endometriosis therapy is administered or continued to be administered to the subject, and/or an RSPO3 inhibitor and/or an RSPO3 Receptor blocker is administered to the subject. An increase in the expression of RSPO3 mRNA or protein indicates the subject has an increased risk of developing endometriosis. In some embodiments, the subject has an increase in the expression of RSPO3 mRNA or protein. In some embodiments, the subject does not have an increase in the expression of RSPO3 mRNA or protein.

The present disclosure also provides methods of preventing a subject from developing endometriosis by administering an endometriosis therapeutic agent or endometriosis therapy that prevents endometriosis. In some embodiments, the method comprises determining whether the subject has an increase in the expression of RSPO3 mRNA or protein by obtaining or having obtained a biological sample from the subject and performing or having performed an assay on the biological sample to determine if the subject has an increase in the expression of RSPO3 mRNA or protein. When the subject does not have an increase in the expression of RSPO3 mRNA or protein, the endometriosis therapeutic agent in a standard dosage amount or endometriosis therapy is administered or continued to be administered to the subject. When the subject has an increase in the expression of RSPO3 mRNA or protein, the endometriosis therapeutic agent in an amount that is the same as or less than a standard dosage amount or endometriosis therapy is administered or continued to be administered to the subject, and/or an RSPO3 inhibitor and/or an RSPO3 Receptor blocker is administered to the subject. The presence of an increase in the expression of RSPO3 mRNA or protein indicates the subject has an increased risk of developing endometriosis. In some embodiments, the subject has an increase in the expression of RSPO3 mRNA or protein. In some embodiments, the subject does not have an increase in the expression of RSPO3 mRNA or protein.

Detecting the presence or absence of an increase in the expression of RSPO3 mRNA or protein in a biological sample from a subject and/or determining whether a subject has an increase in the expression of RSPO3 mRNA or protein can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the RSPO3 inhibitor is a small molecule. In some embodiments, the small molecule is low molecular weight (<900 daltons) organic compound.

In some embodiments, the RSPO3 inhibitor comprises an antibody, or antigen-binding fragment thereof. In some embodiments, the antibody, or antigen-binding fragment thereof, binds specifically to human RSPO3. In some embodiments, the antibody is a fully human monoclonal antibody (mAb), or antigen-binding fragment thereof, that specifically binds and neutralizes, inhibits, blocks, abrogates, reduces, or interferes with, at least one activity of RSPO3, in particular, human RSPO3. In some embodiments, an antibody or fragment thereof can neutralize, inhibit, block, abrogate, reduce, or interfere with, an activity of RSPO3 by binding to an epitope of RSPO3 that is directly involved in the targeted activity of RSPO3. In some embodiments, an antibody or fragment thereof can neutralize, inhibit, block, abrogate, reduce, or interfere with, an activity of RSPO3 by binding to an epitope of RSPO3 that is not directly involved in the targeted activity of RSPO3, but the antibody or fragment binding thereto sterically or conformationally inhibits, blocks, abrogates, reduces, or interferes with, the targeted activity of RSPO3. In some embodiments, an antibody or fragment thereof binds to an epitope of RSPO3 that is not directly involved in the targeted activity of RSPO3 (i.e., a non-blocking antibody), but the antibody or fragment binding thereto results in the enhancement of the clearance of RSPO3 from the circulation, compared to the clearance of RSPO3 in the absence of the antibody or fragment thereof, thereby indirectly inhibiting, blocking, abrogating, reducing, or interfering with, an activity of RSPO3. Clearance of RSPO3 from the circulation can be particularly enhanced by combining two or more different non-blocking antibodies that do not compete with one another for specific binding to RSPO3. The antibodies can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy et al., J. Immunol., 2000, 164, 1925-1933). In some embodiments, the RSPO3 inhibitor comprises an antibody that decreases expression of the RSPO3 protein. In some embodiments, the anti-RSPO3 antibody is rosmantuzumab (OMP-131R10).

In some embodiments, the antibody or antigen-binding fragment thereof specifically binds to RSPO3 with an equilibrium dissociation constant (K_{D}) of about 7 nM or less, about 6 nM or less, about 5 nM or less, about 4 nM or less, about 3 nM or less, about 2 nM or less, or about 1 nM or less, as measured by surface plasmon resonance assay (for example, BIACORE™). In some embodiments, the antibody exhibits a K_{D} of about 800 pM or less, about 700 pM or less; about 600 pM or less; about 500 pM or less; about 400 pM or less; about 300 pM or less; about 200 pM or less; about 100 pM or less; or about 50 pM or less.

In some embodiments, the anti-RSPO3 antibodies have a modified glycosylation pattern. In some applications, modification to remove undesirable glycosylation sites may be useful, or e.g., removal of a fucose moiety to increase antibody dependent cellular cytotoxicity (ADCC) function (see, Shield et al., J. Biol. Chem., 2002, 277, 26733). In other applications, removal of N-glycosylation site may reduce undesirable immune reactions against the therapeutic antibodies or increase affinities of the antibodies. In yet other applications, modification of galactosylation can be made in order to modify complement dependent cytotoxicity (CDC).

The RSPO3 Receptors are leucine-rich repeat-containing G protein-coupled receptor 4 (LGR4), leucine-rich repeat-containing G protein-coupled receptor 5 (LGR5), and leucine-rich repeat-containing G protein-coupled receptor 6 (LGR6). In some embodiments, the RSPO3 Receptor blocker comprises an antibody, or antigen-binding fragment thereof, directed to LGR4, LGR5, and/or LGR6. In some embodiments, the antibody, or antigen-binding fragment thereof, binds specifically to human LGR4, LGR5, and/or LGR6. In some embodiments, the antibody is a fully human monoclonal antibody (mAb), or antigen-binding fragment thereof, that specifically binds and neutralizes, inhibits, blocks, abrogates, reduces, or interferes with, at least one activity of LGR4, LGR5, and/or LGR6, in particular, human LGR4, LGR5, and/or LGR6. In some embodiments, an antibody or fragment thereof can neutralize, inhibit, block, abrogate, reduce, or interfere with, an activity of LGR4, LGR5, and/or LGR6 by binding to an epitope of LGR4, LGR5, and/or LGR6 that is directly involved in the targeted activity of LGR4, LGR5, and/or LGR6. In some embodiments, an antibody or fragment thereof can neutralize, inhibit, block, abrogate, reduce, or interfere with, an activity of LGR4, LGR5, and/or LGR6 by binding to an epitope of LGR4, LGR5, and/or LGR6 that is not directly involved in the targeted activity of LGR4, LGR5, and/or LGR6, but the antibody or fragment binding thereto sterically or conformationally inhibits, blocks, abrogates, reduces, or interferes with, the targeted activity of LGR4, LGR5, and/or LGR6. The antibodies can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy et al., J. Immunol., 2000, 164, 1925-1933). In some embodiments, the RSPO3 Receptor blocker comprises an antibody that decreases expression of the LGR4, LGR5, and/or LGR6 protein. In some embodiments, the RSPO3 Receptor blocker comprises an antibody directed to LGR4 (see, for example, WO 2015/200073).

In some embodiments, the antibody or antigen-binding fragment thereof specifically binds to LGR4, LGR5, and/or LGR6 with an equilibrium dissociation constant (K D) of about 7 nM or less, about 6 nM or less, about 5 nM or less, about 4 nM or less, about 3 nM or less, about 2 nM or less, or about 1 nM or less, as measured by surface plasmon resonance assay (for example, BIACORE™). In some embodiments, the antibody exhibits a K_{D} of about 800 pM or less, about 700 pM or less; about 600 pM or less; about 500 pM or less; about 400 pM or less; about 300 pM or less; about 200 pM or less; about 100 pM or less; or about 50 pM or less.

In some embodiments, the LGR4, LGR5, and/or LGR6 antibodies have a modified glycosylation pattern. In some applications, modification to remove undesirable glycosylation sites may be useful, or e.g., removal of a fucose moiety to increase antibody dependent cellular cytotoxicity (ADCC) function (see, Shield et al., J. Biol. Chem., 2002, 277, 26733). In other applications, removal of N-glycosylation site may reduce undesirable immune reactions against the therapeutic antibodies or increase affinities of the antibodies. In yet other applications, modification of galactosylation can be made in order to modify complement dependent cytotoxicity (CDC).

The present disclosure also provides compositions comprising a combination of an antibody or antigen-binding fragment thereof and an endometriosis therapeutic agent.

In some embodiments, the endometriosis therapeutic agents may include, but are not limited to, analgesics (e.g., non-steroidal inflammatory drugs (NSAIDS) such as ibuprofen and maproxen), hormones (e.g., estrogen and progestin), gonadotropin-releasing hormone (GnRH) analogues, which may be GnRH agonists (e.g., goserelin, leuprolide, and nafarelin) or antagonists (e.g., elagolix), androgen receptor agonists (e.g., danazol) and aromatase inhibitors. Additional endometriosis therapies include any therapy used to reduce or manage endometriosis risk factors. In some embodiments, the endometriosis therapeutic agent or endometriosis therapy can be combined with an RSPO3 inhibitor and/or an RSPO3 Receptor blocker. In some embodiments, the treatment therapy for endometriosis may include surgery to remove endometrial implants, drain endometrial cysts, and/or remove the cyst wall. These treatment therapies may be delayed or avoided altogether by treatment with an RSPO3 inhibitor and/or an RSPO3 Receptor blocker as described herein.

In some embodiments, the dose of the endometriosis therapeutic agents that treat, prevent, or inhibit endometriosis can be decreased by about 10%, by about 20%, by about 30%, by about 40%, by about 50%, by about 60%, by about 70%, by about 80%, or by about 90% for subjects that are heterozygous or homozygous for an RSPO3 variant nucleic acid molecule (i.e., a less than the standard dosage amount) compared to subjects that are RSPO3 reference (who may receive a standard dosage amount). In some embodiments, the dose of the endometriosis therapeutic agents that treat, prevent, or inhibit endometriosis can be decreased by about 10%, by about 20%, by about 30%, by about 40%, or by about 50%. In addition, the subjects that are heterozygous or homozygous for an RSPO3 variant nucleic acid molecule can be administered less frequently compared to subjects that are RSPO3 reference.

In some embodiments, the dose of the endometriosis therapeutic agents that treat, prevent, or inhibit endometriosis can be decreased by about 10%, by about 20%, by about 30%, by about 40%, by about 50%, for subjects that are homozygous for an RSPO3 variant nucleic acid molecule compared to subjects that are heterozygous for an RSPO3 variant nucleic acid molecule. In some embodiments, the dose of the endometriosis therapeutic agents can be decreased by about 10%, by about 20%, by about 30%, by about 40%, or by about 50%. In addition, the dose of endometriosis therapeutic agents in subjects that are homozygous for an RSPO3 variant nucleic acid molecule can be administered less frequently compared to subjects that are heterozygous for an RSPO3 variant nucleic acid molecule.

Administration of the endometriosis therapeutic agents that treat, prevent, or inhibit endometriosis and/or RSPO3 inhibitors and/or RSPO3 Receptor blockers can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the endometriosis therapeutic agents that treat, prevent, or inhibit endometriosis and/or RSPO3 inhibitors and/or RSPO3 Receptor blockers can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients, or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in endometriosis, a decrease/reduction in the severity of endometriosis (such as, for example, a reduction or inhibition of development of endometriosis), a decrease/reduction in symptoms and disease-related effects, delaying the onset of symptoms and disease-related effects, reducing the severity of symptoms of disease-related effects, reducing the number of symptoms and disease-related effects, reducing the latency of symptoms and disease-related effects, an amelioration of symptoms and disease-related effects, reducing secondary symptoms, reducing secondary infections, preventing relapse to endometriosis, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of endometriosis development/progression (such as, for example, a complete or partial avoidance/inhibition or a delay), and an increased survival time of the affected host animal, following administration of a therapeutic protocol. Treatment of endometriosis encompasses the treatment of a subject already diagnosed as having any form of endometriosis at any clinical stage or manifestation, the delay of the onset or evolution or aggravation or deterioration of the symptoms or signs of endometriosis, and/or preventing and/or reducing the severity of endometriosis.

In some embodiments, the RSPO3 inhibitor and/or the RSPO3 Receptor blocker and the endometriosis therapeutic agent are disposed within a pharmaceutical composition. In some embodiments, the RSPO3 inhibitor and/or the RSPO3 Receptor blocker is disposed within a first pharmaceutical composition and the endometriosis therapeutic agent is disposed within a second pharmaceutical composition. In some embodiments, the first pharmaceutical composition and the second pharmaceutical composition are administered simultaneously. In some embodiments, the first pharmaceutical composition is administered before the second pharmaceutical composition. In some embodiments, the first pharmaceutical composition is administered after the second pharmaceutical composition.

In some embodiments, the RSPO3 variant nucleic acid molecule comprises any one or more of the following genetic variations: 6:127125465:G:C (r59482771), 6:126727085:C:T (r5111743285), 6:126973497:T:C (r59398828), 6:127017612:T:C (r5117698897), 6:127114919:C:T (r51936800), 6:127118748:C:G (r5138650988), 6:127118902:T:C (r5577721086), 1:19979653:G:C (r511573156), 3:56815721:T:C (r51354034), 3:136318144:T:C (r5664088), 6:2507158:T:C (r5879097), 6:117184879:T:G (r5200564310), 6:154441670:T:C (r575045626), 6:160671406:C:T (r511751347), 6:160706469:A:G (r573015965), 6:160716958:G:A (r5783147), 6:160731873:C:T (r54252129), 6:160741337:T:A (r5147175166), 7:151716108:T:C (r510224210), 8:105578477:C:A (r54541868), 12:71474678:T:C (r560195346), 13:73551661:G:A (r51887646), 14:54766806:G:A (r57147275), 14:54768564:C:T (r54901541), 14:94371805:G:T (r5112635299), 16:251942:A:G (rs9934955), 16:3697041:G:T (r51635404), 17:28367840:G:A (rs704), 19:44846145:T:C (r53810143), 19:55027612:T:C (r51654425), 20:45347027:A:C (r51981430), 20:45354752:G:A (r56065808), and 23:70475947:G:T. In some embodiments, the RSPO3 variant nucleic acid molecule comprises any one or more of the following genetic variations: 6:127125465:G:C (r59482771), 6:126727085:C:T (r5111743285), 6:126973497:T:C (r59398828), 6:127017612:T:C (r5117698897), 6:127114919:C:T (r51936800), 6:127118748:C:G (r5138650988), and 6:127118902:T:C (r5577721086). In some embodiments, the RSPO3 variant nucleic acid molecule comprises the genetic variation 6:127125465:G:C (r59482771).

The present disclosure also provides methods of identifying a subject having an increased risk of developing endometriosis. In some embodiments, the method comprises determining or having determined in a biological sample obtained from the subject the presence or absence of an RSPO3 variant nucleic acid molecule (such as a genomic nucleic acid molecule, mRNA molecule, and/or cDNA molecule). When the subject lacks an RSPO3 variant nucleic acid molecule (i.e., the subject is genotypically categorized as RSPO3 reference), then the subject does not have an increased risk of developing endometriosis. When the subject has an RSPO3 variant nucleic acid molecule (i.e., the subject is heterozygous or homozygous for an RSPO3 variant nucleic acid molecule), then the subject has an increased risk of developing endometriosis. In some embodiments, the RSPO3 variant nucleic acid molecule comprises any one or more of the following genetic variations: 6:127125465:G:C (r59482771), 6:126727085:C:T (r5111743285), 6:126973497:T:C (r59398828), 6:127017612:T:C (r5117698897), 6:127114919:C:T (r51936800), 6:127118748:C:G (r5138650988), 6:127118902:T:C (r5577721086), 1:19979653:G:C (r511573156), 3:56815721:T:C (r51354034), 3:136318144:T:C (r5664088), 6:2507158:T:C (r5879097), 6:117184879:T:G (r5200564310), 6:154441670:T:C (r575045626), 6:160671406:C:T (r511751347), 6:160706469:A:G (r573015965), 6:160716958:G:A (r5783147), 6:160731873:C:T (r54252129), 6:160741337:T:A (r5147175166), 7:151716108:T:C (r510224210), 8:105578477:C:A (r54541868), 12:71474678:T:C (r560195346), 13:73551661:G:A (r51887646), 14:54766806:G:A (r57147275), 14:54768564:C:T (r54901541), 14:94371805:G:T (r5112635299), 16:251942:A:G (rs9934955), 16:3697041:G:T (r51635404), 17:28367840:G:A (rs704), 19:44846145:T:C (r53810143), 19:55027612:T:C (r51654425), 20:45347027:A:C (r51981430), 20:45354752:G:A (r56065808), and 23:70475947:G:T. In some embodiments, the RSPO3 variant nucleic acid molecule comprises any one or more of the following genetic variations: 6:127125465:G:C (r59482771), 6:126727085:C:T (r5111743285), 6:126973497:T:C (r59398828), 6:127017612:T:C (r5117698897), 6:127114919:C:T (r51936800), 6:127118748:C:G (r5138650988), and 6:127118902:T:C (r5577721086). In some embodiments, the RSPO3 variant nucleic acid molecule comprises the genetic variation 6:127125465:G:C (r59482771).

Having two copies of an RSPO3 variant nucleic acid molecule may indicate a greater risk of developing endometriosis than having one copy of an RSPO3 variant nucleic acid molecule. Without intending to be limited to any particular theory or mechanism of action, it is believed that having two copies of an RSPO3 variant nucleic acid molecule (i.e., homozygous for an RSPO3 variant nucleic acid molecule) may indicate a greater risk of developing endometriosis and it is also believed that having a single copy of an RSPO3 variant nucleic acid molecule (i.e., heterozygous for an RSPO3 variant nucleic acid molecule) may indicate a greater risk of developing endometriosis, relative to a subject with no copies. While not desiring to be bound by any particular theory, there may be additional factors or molecules involved in the development of endometriosis that are still present in a subject having a single copy of an RSPO3 variant nucleic acid molecule.

Determining whether a subject has an RSPO3 variant nucleic acid molecule in a biological sample from a subject and/or determining whether a subject has an RSPO3 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing endometriosis, the subject is administered an endometriosis therapeutic agent or endometriosis therapy, and/or an RSPO3 inhibitor, and/or an RSPO3 Receptor blocker, as described herein. For example, when the subject is heterozygous or homozygous for an RSPO3 variant nucleic acid molecule, and therefore has an increased risk of developing endometriosis, the subject is administered an endometriosis therapeutic agent or endometriosis therapy, and/or is administered an RSPO3 inhibitor and/or an RSPO3 Receptor blocker. In some embodiments, when the subject is heterozygous or homozygous for an RSPO3 variant nucleic acid molecule, the subject is administered the endometriosis therapeutic agent in a dosage amount that is the same as or less than a standard dosage amount or endometriosis therapy, and/or is administered an RSPO3 inhibitor, and/or an RSPO3 Receptor blocker. In some embodiments, when the subject is RSPO3 reference, the subject is administered the endometriosis therapeutic agent in a standard dosage amount or endometriosis therapy. In some embodiments, the subject is RSPO3 reference. In some embodiments, the subject is heterozygous or homozygous for an RSPO3 variant nucleic acid molecule.

The present disclosure also provides methods of determining a subject's aggregate burden, or risk score, of having two or more RSPO3 variant nucleic acid molecules associated with an increased risk of developing endometriosis. The aggregate burden is the sum of two or more genetic variants that can be carried out in an association analysis with endometriosis. In some embodiments, the subject is homozygous for one or more RSPO3 variant nucleic acid molecules associated with an increased risk of developing endometriosis. In some embodiments, the subject is heterozygous for one or more RSPO3 variant nucleic acid molecules associated with an increased risk of developing endometriosis. When the subject has a higher aggregate burden, the subject is at a greater risk of developing endometriosis and the subject is administered or continued to be administered the endometriosis therapeutic agent in an amount that is the same as or less than the standard dosage amount or endometriosis therapy, and/or an RSPO3 inhibitor, and/or an RSPO3 Receptor blocker. When the subject has a lower aggregate burden, the subject is at a lesser risk of developing endometriosis and the subject is administered or continued to be administered the endometriosis therapeutic agent in a standard amount or endometriosis therapy. The higher the aggregate burden, the greater the risk of developing endometriosis.

In some embodiments, a subject's aggregate burden of having any two or more RSPO3 variant nucleic acid molecules represents a weighted sum of a plurality of any of the RSPO3 variant nucleic acid molecules. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the RSPO3 gene, where the genetic burden is the number of alleles multiplied by the association estimate with endometriosis or related outcome for each allele (e.g., a weighted polygenic burden score). In some embodiments, when the subject has an aggregate burden above a desired threshold score, the subject has an increased risk of developing endometriosis. In some embodiments, when the subject has an aggregate burden below a desired threshold score, the subject has a decreased risk of developing endometriosis.

In some embodiments, the aggregate burden may be divided into quintiles, e.g., top quintile, second quintile, intermediate quintile, fourth quintile, and bottom quintile, wherein the top quintile of aggregate burden corresponds to the greatest risk group and the bottom quintile of aggregate burden corresponds to the lowest risk group. In some embodiments, a subject having a greater aggregate burden comprises the highest weighted aggregate burdens, including, but not limited to the top 10%, top 20%, top 30%, top 40%, or top 50% of aggregate burdens from a subject population. In some embodiments, the genetic variants comprise the genetic variants having association with endometriosis in the top 10%, top 20%, top 30%, top 40%, or top 50% of p-value range for the association. In some embodiments, each of the identified genetic variants comprise the genetic variants having association with endometriosis with p-value of no more than about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with endometriosis with p-value of less than 5×10⁻⁸. In some embodiments, the identified genetic variants comprise genetic variants having association with endometriosis in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects comprise subjects having aggregate burdens in the top decile, quintile, or tertile in a reference population. The threshold of the aggregate burden is determined on the basis of the nature of the intended practical application and the risk difference that would be considered meaningful for that practical application.

In embodiments where the aggregate burden is determined for RSPO3 genetic variants associated with endometriosis, then the aggregate burden represents a subject's risk score for developing endometriosis. In some embodiments, the aggregate burden or risk score includes the RSPO3 variant genomic nucleic acid molecule that comprises the genetic variation rs9482771, or is an mRNA molecule produced therefrom, or is a cDNA molecule produced from the mRNA molecule. In some embodiments, a subject's aggregate burden can be determined for RSPO3 genetic variants associated with endometriosis in combination with additional genetic variants for other genes also associated with endometriosis to produce a polygenic risk score (PRS) for developing endometriosis. In some embodiments, the PRS includes the RSPO3 variant genomic nucleic acid molecule that comprises the genetic variation rs9482771, or is an mRNA molecule produced therefrom, or is a cDNA molecule produced from the mRNA molecule.

The present disclosure also provides methods of detecting the presence or absence of an RSPO3 variant nucleic acid molecule (i.e., a genomic nucleic acid molecule, an mRNA molecule, or a cDNA molecule produced from an mRNA molecule) in a biological sample from a subject. It is understood that gene sequences within a population and mRNA molecules encoded by such genes can vary due to polymorphisms such as single-nucleotide polymorphisms.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any RSPO3 variant nucleic acid molecule, preliminary processing designed to isolate or enrich the biological sample for the genomic DNA can be employed. A variety of techniques may be used for this purpose. When detecting the level of any RSPO3 variant nucleic acid molecule, different techniques can be used enrich the biological sample with mRNA molecules. Various methods to detect the presence or level of an mRNA molecule or the presence of a particular variant genomic DNA locus can be used.

In some embodiments, detecting an RSPO3 variant nucleic acid molecule in a subject comprises performing a sequence analysis on a biological sample obtained from the subject to determine whether an RSPO3 genomic nucleic acid molecule in the biological sample, and/or an RSPO3 mRNA molecule in the biological sample, and/or an RSPO3 cDNA molecule produced from an mRNA molecule in the biological sample, comprises one or more variations that cause a loss-of-function (partial or complete) or are predicted to cause a loss-of-function (partial or complete). In some embodiments, the methods detect the RSPO3 variant genomic nucleic acid molecule that comprises any one or more of the following genetic variations: 6:127125465:G:C (r59482771), 6:126727085:C:T (r5111743285), 6:126973497:T:C (r59398828), 6:127017612:T:C (r5117698897), 6:127114919:C:T (r51936800), 6:127118748:C:G (r5138650988), 6:127118902:T:C (r5577721086), 1:19979653:G:C (r511573156), 3:56815721:T:C (r51354034), 3:136318144:T:C (r5664088), 6:2507158:T:C (r5879097), 6:117184879:T:G (r5200564310), 6:154441670:T:C (r575045626), 6:160671406:C:T (r511751347), 6:160706469:A:G (r573015965), 6:160716958:G:A (r5783147), 6:160731873:C:T (r54252129), 6:160741337:T:A (r5147175166), 7:151716108:T:C (r510224210), 8:105578477:C:A (r54541868), 12:71474678:T:C (r560195346), 13:73551661:G:A (r51887646), 14:54766806:G:A (r57147275), 14:54768564:C:T (r54901541), 14:94371805:G:T (r5112635299), 16:251942:A:G (rs9934955), 16:3697041:G:T (r51635404), 17:28367840:G:A (rs704), 19:44846145:T:C (r53810143), 19:55027612:T:C (r51654425), 20:45347027:A:C (r51981430), 20:45354752:G:A (r56065808), and 23:70475947:G:T. In some embodiments, the methods detect the RSPO3 variant genomic nucleic acid molecule that comprises any one or more of the following genetic variations: 6:127125465:G:C (r59482771), 6:126727085:C:T (r5111743285), 6:126973497:T:C (r59398828), 6:127017612:T:C (r5117698897), 6:127114919:C:T (r51936800), 6:127118748:C:G (r5138650988), and 6:127118902:T:C (r5577721086). In some embodiments, the RSPO3 variant nucleic acid molecule comprises the genetic variation 6:127125465:G:C (rs9482771). In some embodiments, the methods detect the RSPO3 variant genomic nucleic acid molecule that comprises the genetic variation rs9482771, or an mRNA molecule produced therefrom, or a cDNA molecule produced from the mRNA molecule.

In some embodiments, the methods of detecting the presence or absence of an Rspo3 variant nucleic acid molecule (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising an RSPO3 genomic nucleic acid molecule or mRNA molecule, and if mRNA, optionally reverse transcribing the mRNA into cDNA. Such assays can comprise, for example determining the identity of these positions of the particular RSPO3 nucleic acid molecule. In some embodiments, the method is an in vitro method.

In some embodiments, the determining step, detecting step, or sequence analysis comprises sequencing at least a portion of the nucleotide sequence of the RSPO3 genomic nucleic acid molecule, the RSPO3 mRNA molecule, or the RSPO3 cDNA molecule in the biological sample, wherein the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only genomic nucleic acid molecules, such as an RSPO3 genomic nucleic acid molecule, is analyzed. In some embodiments, only mRNA molecules, such as an RSPO3 mRNA is analyzed. In some embodiments, only cDNA molecules, such as an Rspo3 cDNA obtained from the RSPO3 mRNA, is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to an RSPO3 variant genomic sequence, variant mRNA sequence, or variant cDNA sequence and not the corresponding RSPO3 reference sequence under stringent conditions and determining whether hybridization has occurred.

In some embodiments, the determining step, detecting step, or sequence analysis comprises: a) amplifying at least a portion of the RSPO3 nucleic acid molecule; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an RSPO3 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to RSPO3 variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to RSPO3 variant nucleic acid molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the RSPO3 variant nucleic acid molecules disclosed herein. The primers described herein can be used to amplify any RSPO3 variant nucleic acid molecule, or a fragment thereof.

In the context of the disclosure “specifically hybridizes” means that the probe or primer (such as, for example, the alteration-specific probe or alteration-specific primer) does not hybridize to an RSPO3 reference genomic nucleic acid molecule, an RSPO3 reference mRNA molecule, and/or an RSPO3 reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×his or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides endometriosis therapeutic agents that treat, prevent, or inhibit endometriosis for use in the treatment or prevention of endometriosis in a subject having an RSPO3 variant nucleic acid molecule. Any of the endometriosis therapeutic agents that treat, prevent, or inhibit endometriosis described herein can be used in these methods. Any of the RSPO3 variant nucleic acid molecules disclosed herein can be used. In some embodiments, the RSPO3 variant nucleic acid molecule is an RSPO3 variant genomic nucleic acid molecule that comprises the genetic variation rs9482771, or is an mRNA molecule produced therefrom, or is a cDNA molecule produced from the mRNA molecule.

The present disclosure also provides uses of endometriosis therapeutic agents that treat, prevent, or inhibit endometriosis for use in the preparation of a medicament for treating and/or preventing endometriosis in a subject having an RSPO3 variant nucleic acid molecule. Any of the endometriosis therapeutic agents that treat, prevent, or inhibit endometriosis described herein can be used in these methods. Any of the RSPO3 variant nucleic acid molecules disclosed herein can be used. In some embodiments, the RSPO3 variant nucleic acid molecule is an RSPO3 variant genomic nucleic acid molecule that comprises the genetic variation rs9482771, or is an mRNA molecule produced therefrom, or is a cDNA molecule produced from the mRNA molecule.

The present disclosure also provides RSPO3 inhibitors and/or RSPO3 Receptor blockers for use in the treatment or prevention of endometriosis in a subject that is heterozygous or homozygous for an RSPO3 variant nucleic acid molecule. Any of the RSPO3 inhibitors and/or RSPO3 Receptor blockers described herein can be used. Any of the RSPO3 variant nucleic acid molecules disclosed herein can be used. In some embodiments, the RSPO3 variant nucleic acid molecule is an RSPO3 variant genomic nucleic acid molecule that comprises the genetic variation rs9482771, or is an mRNA molecule produced therefrom, or is a cDNA molecule produced from the mRNA molecule.

The present disclosure also provides RSPO3 inhibitors and/or RSPO3 Receptor blockers in the preparation of a medicament for treating and/or preventing endometriosis in a subject that is heterozygous or homozygous for an RSPO3 variant nucleic acid molecule. Any of the RSPO3 inhibitors and/or RSPO3 Receptor blockers described herein can be used. Any of the RSPO3 variant nucleic acid molecules disclosed herein can be used. In some embodiments, the RSPO3 variant nucleic acid molecule is an RSPO3 variant genomic nucleic acid molecule that comprises the genetic variation rs9482771, or is an mRNA molecule produced therefrom, or is a cDNA molecule produced from the mRNA molecule.

In some embodiments, the RSPO3 inhibitor and/or the RSPO3 Receptor blocker, and the endometriosis therapeutic agent are disposed within a pharmaceutical composition. In some embodiments, the RSPO3 inhibitor and/or the RSPO3 Receptor blocker is disposed within a first pharmaceutical composition and the endometriosis therapeutic agent is disposed within a second pharmaceutical composition. In some embodiments, the first pharmaceutical composition and the second pharmaceutical composition are administered simultaneously. In some embodiments, the first pharmaceutical composition is administered before the second pharmaceutical composition. In some embodiments, the first pharmaceutical composition is administered after the second pharmaceutical composition.

In some embodiments, the RSPO3 variant nucleic acid molecule comprises a splice-site variant, a stop-gain variant, a start-loss variant, a stop-loss variant, a frameshift variant, or an in-frame indel variant. In some embodiments, the RSPO3 variant nucleic acid molecule comprises any one or more of the following genetic variations: 6:127125465:G:C (r59482771), 6:126727085:C:T (r5111743285), 6:126973497:T:C (r59398828), 6:127017612:T:C (r5117698897), 6:127114919:C:T (r51936800), 6:127118748:C:G (r5138650988), 6:127118902:T:C (r5577721086), 1:19979653:G:C (r511573156), 3:56815721:T:C (r51354034), 3:136318144:T:C (r5664088), 6:2507158:T:C (r5879097), 6:117184879:T:G (r5200564310), 6:154441670:T:C (r575045626), 6:160671406:C:T (r511751347), 6:160706469:A:G (r573015965), 6:160716958:G:A (r5783147), 6:160731873:C:T (r54252129), 6:160741337:T:A (r5147175166), 7:151716108:T:C (r510224210), 8:105578477:C:A (r54541868), 12:71474678:T:C (r560195346), 13:73551661:G:A (r51887646), 14:54766806:G:A (r57147275), 14:54768564:C:T (r54901541), 14:94371805:G:T (r5112635299), 16:251942:A:G (rs9934955), 16:3697041:G:T (r51635404), 17:28367840:G:A (rs704), 19:44846145:T:C (r53810143), 19:55027612:T:C (r51654425), 20:45347027:A:C (r51981430), 20:45354752:G:A (r56065808), and 23:70475947:G:T. In some embodiments, the RSPO3 variant nucleic acid molecule comprises any one or more of the following genetic variations: 6:127125465:G:C (r59482771), 6:126727085:C:T (r5111743285), 6:126973497:T:C (r59398828), 6:127017612:T:C (r5117698897), 6:127114919:C:T (r51936800), 6:127118748:C:G (r5138650988), and 6:127118902:T:C (r5577721086). In some embodiments, the RSPO3 variant nucleic acid molecule comprises the genetic variation 6:127125465:G:C (r59482771).

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Variant (r59482771) is Significantly Associated (Genome-Wide) in the Meta-Analysis with Endometriosis

Genome-wide association analysis for endometriosis was conducted in 8 cohorts, and the results were meta-analysed to identify genetic variants associated with disease risk. Both common variants and rare variants were analysed in burden tests.

Separately, genetic association analysis was performed for RSPO3 protein levels (a pQTL study), in UKB, to identify genetic variants which influence the circulating levels of RSPO3. The variants associated with RSPO3 levels were used in a mendelian randomization analysis to show that RSPO3 levels are causally involved in endometriosis.

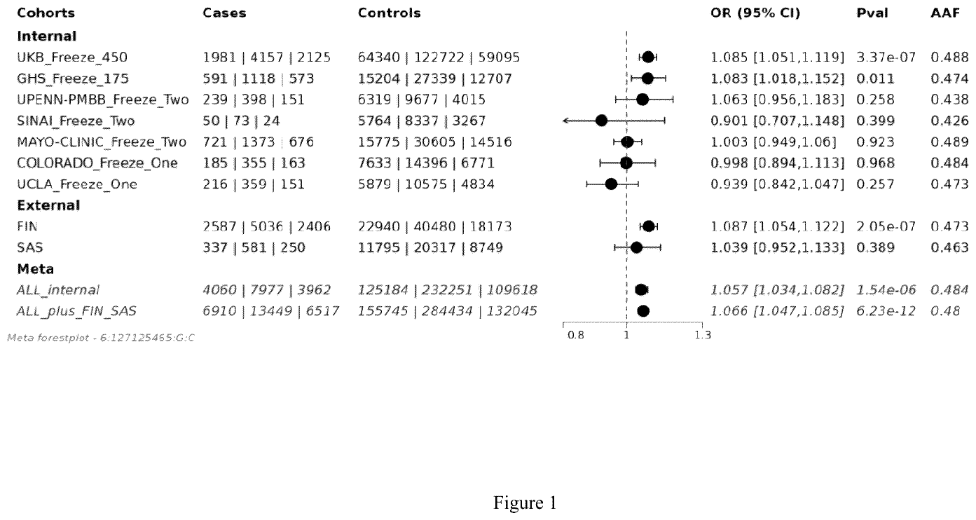

A study was carried out to determine the association of Index Variant 6:127125465:G:C with endometriosis (see, Table 1, Table 2, and FIG. 1).

TABLE 1

| Effect (CI) | P-value | Cases | Controls | AAF |
|---|---|---|---|---|
| 1.066 | 6.2 × 10⁻¹² | 6910\|13449\|6517 | 155745\|284434\|132045 | 0.480 |
| (1.047, 1.085) | | | | |

TABLE 2
RSPO3 Gene

| Index | Effect | | | | |
|---|---|---|---|---|---|
| Variant | (CI) | P-value | Cases | Controls | AAF |
| 1 | 0.896 | 0.861 | 14517\|1\|0 | 403276\|68\|0 | 9.00E−05 |
| | (0.261, | | | | |
| | 3.078) | | | | |
| 2 | 0.715 | 0.384 | 15706\|5\|0 | 459058\|216\|0 | 0.00025 |
| | (0.337, | | | | |
| | 1.52) | | | | |

Index Variant 1 = pLOF, MAF < 1%
Index Variant 2 = pLOFs and deleterious missense, MAF < 1%

Variant (r59482771) is significantly associated (genome-wide) in the meta-analysis, with endometriosis.

RSPO3 has consistent results between cis and trans estimates, and has the same direction of effect with the trait (i.e., lower levels of protein are associated with lower endometriosis disease) (see, Table 3).

TABLE 3

| | Instruments | | Instruments in | Pleiotropy test (MR-Egger |
|---|---|---|---|---|
| cis OR (CI) | in cis P-value | trans OR (CI) | trans P-value | Intercept P-value) |
| 1.405 | 8.86E−07 | 1.154 | 0.0637 | 0.3181 |
| (1.227, 1.61) | | (0.992, 1.343) | | |

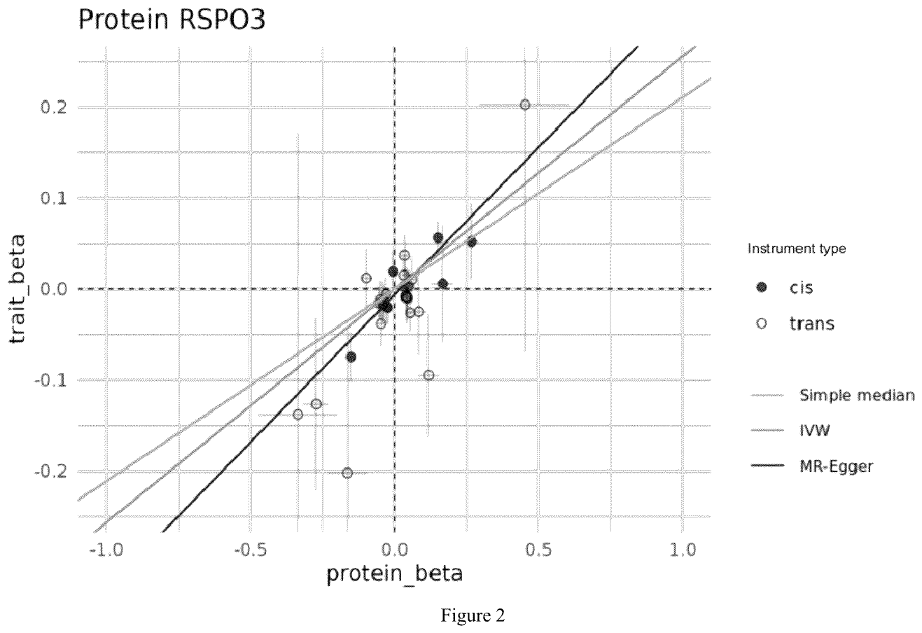

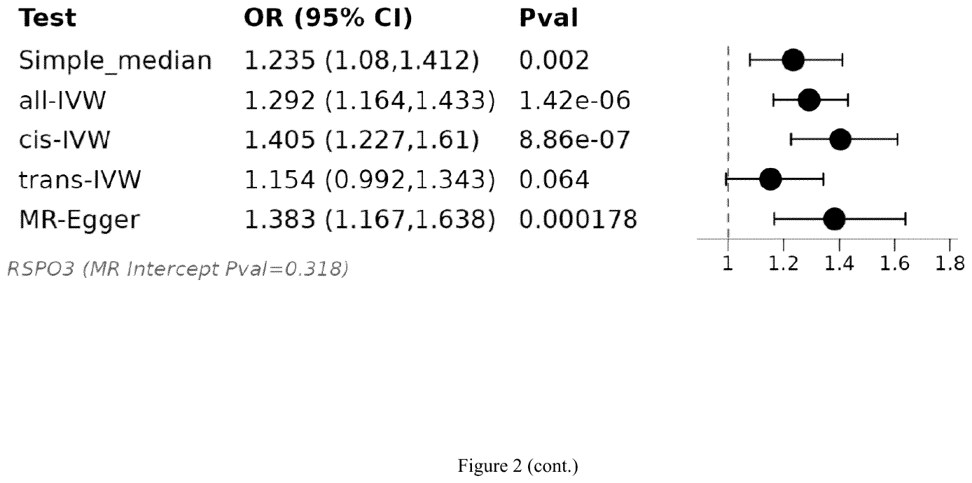

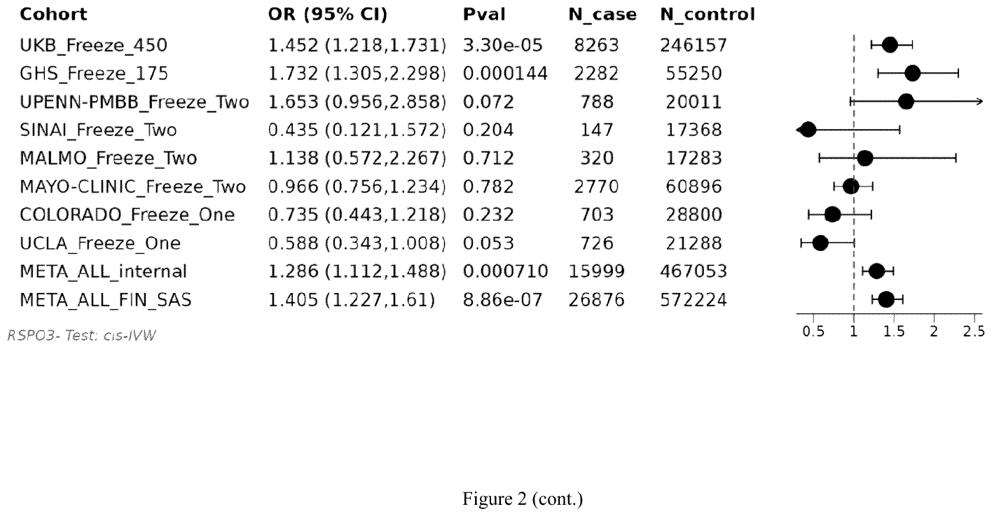

RSPO3 protein levels are associated with endometriosis outcome. Increased protein levels associated with increase in disease risk, when assessed using two MR methods: inverse-weighted variance (IVW) and MR-Egger. The two analytical methods are consistent, and results suggest there is no pleiotropy. The results are driven by cis-variants. The trans-variants span 12 different chromosomes. The MR-estimates are consistent and significant in UKB, GHS and UPENN-PMBB (see, FIG. 2).

RSPO3—sumstats in endometriosis for cis-instruments (variants pQTL data) (see, Table 4).

TABLE 4

| Name | Effect (CI) | P-value | Cases | Controls | AAF |
|---|---|---|---|---|---|
| 1 | 1.058 | 1.24E−09 | 6184\|13484\|7208 | 135555\|285302\|51367 | 0.514 |
| | (1.039, 1.077) | | | | |
| 2 | 0.928 | 1.41E−07 | 20527\|5881\|468 | 425896\|134687\|11641 | 0.138 |
| | (0.903, 0.954) | | | | |
| 3 | 1.053 | 0.016 | 24106\|2693\|77 | 522580\|48340\|1305 | 0.045 |
| | (1.01, 1.099) | | | | |
| 4 | 0.98 | 0.031 | 8323\|13289\|5264 | 177287\|280987\|113949 | 0.445 |
| | (0.962, 0.998) | | | | |
| 5 | 1.019 | 0.104 | 17490\|8291\|1096 | 372625\|176722\|22877 | 0.194 |
| | (0.996, 1.043) | | | | |
| 6 | 1.006 | 0.862 | 25760\|1104\|12 | 550425\|21551\|248 | 0.019 |
| | (0.942, 1.073) | | | | |

1 = 6:127114919:C:T;
2 = 6:127118748:C:G;
3 = 6:127118902:T:C;
4 = 6:126973497:T:C;
5 = 6:126727085:C:T;
6 = 6:127017612:T:C

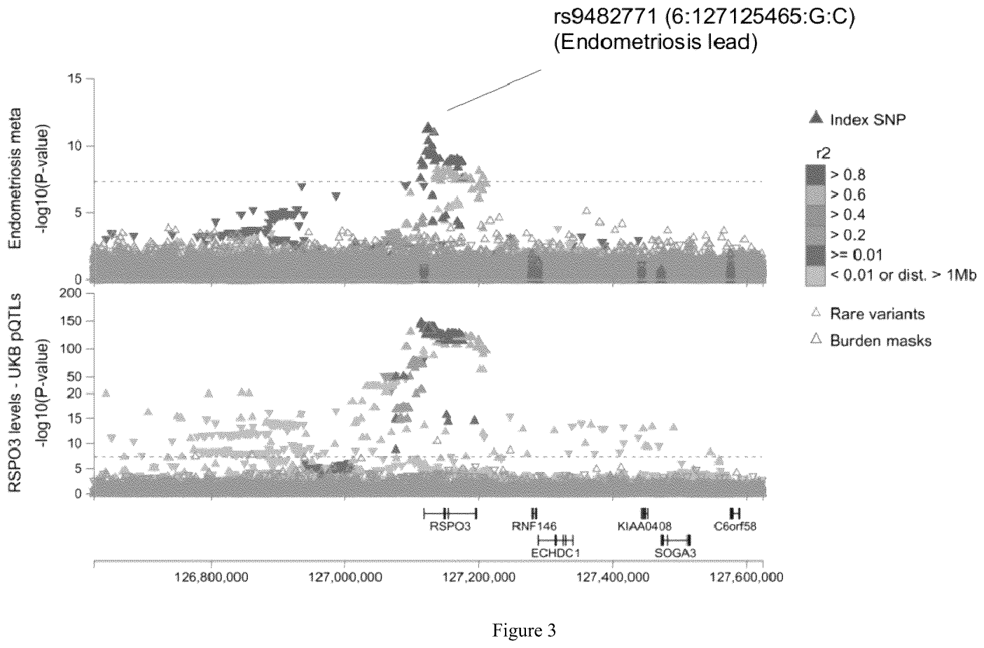

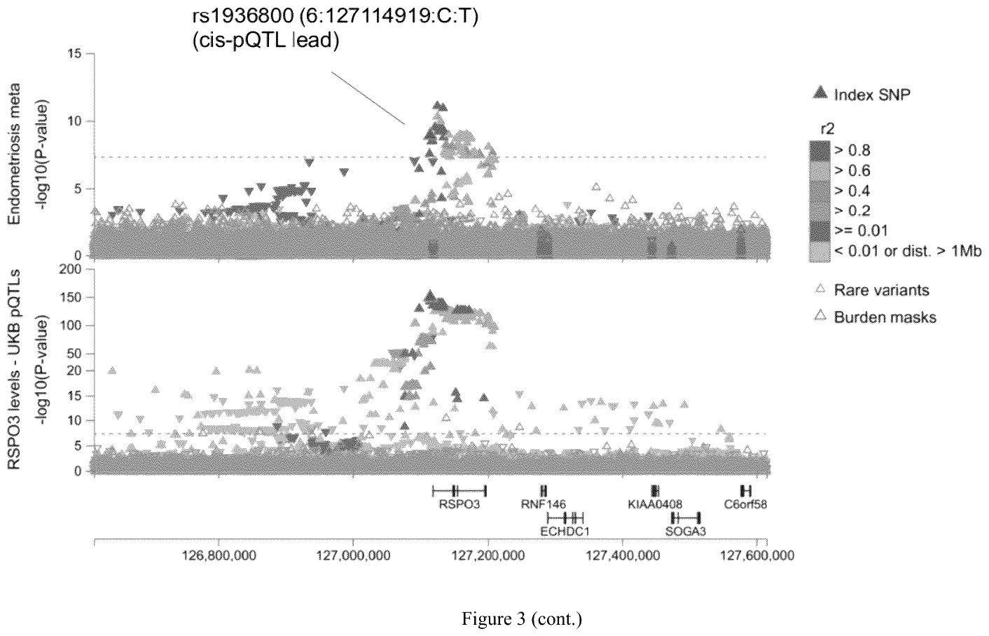

RSPO3—endometriosis signal co-localizes with cis-pQTL signal. Lead variant from endometriosis 6:127125465:G:C is in LD in EUR (UKB) with the cis pQTL signal, confirming colocalization of the signal (see, FIG. 3).

### Example 2: RSPO3 Expression and Other WNT Signaling Genes

Commercially available cryopreserved cells from uterine tissues were acquired from two different vendors. Cells were thawed and lysed directly to avoid gene expression changes that may result from culturing. Additional vials from the vendors have been acquired for single cell experiments. Table 5 shows the cell samples.

TABLE 5

| Vendor | Number of Donors | Uterine Cell Type |
|---|---|---|
| PromoCell | 2 | Endothelial |
| PromoCell | 2 | Fibroblast |
| LifeForce | 4 | Fibroblast |
| LifeForce | 3 | Smooth Muscle |
| LifeForce | 4 | Smooth Muscle |
| LifeForce | 1 | Endometrial epithelial |

All single cells from different uterine samples were pooled and analysed together for single cell analysis. A pool of endothelial (2 individuals), fibroblast (6 individuals), smooth muscle (7), and endometrial epithelial (1 individual) were analysed. Vendor cell annotation (cell type) was confirmed with in-house and published markers for uterine data. Cell types that express RSPO3 and other genes in the same signaling pathway (the WNT pathway—WNT4, LGR5, and AXIN2) were observed.

In particular, RNASeq library for single cell RNA sequencing (scRNA-Seq) was prepared as described in Lin et al. (Commun. Biol., 2023, 6, 447). Single cells were suspended in PBS with 0.04% BSA and were loaded at 10,000 cells per lane on a Chromium Connect Single-Cell Liquid Handler (10× Genomics). RNA-seq libraries were prepared using Chromium Next GEM Automated Single-Cell 5′ Kit, v2 (10× Genomics). Paired-end sequencing was performed on Illumina NovaSeq 6000 for RNA-seq libraries (Read 128 bp for UMI and cell barcode, Read 280-bp for transcript read, with 10-bp i7 and 10-bp i5 reads). Cell Ranger Single-Cell Software Suite (10× Genomics, v2.2.0) was used to perform sample demultiplexing, alignment, filtering, and UMI counting. The human GRCh38 genome assembly and RefSeq gene model for human were used for the alignment.

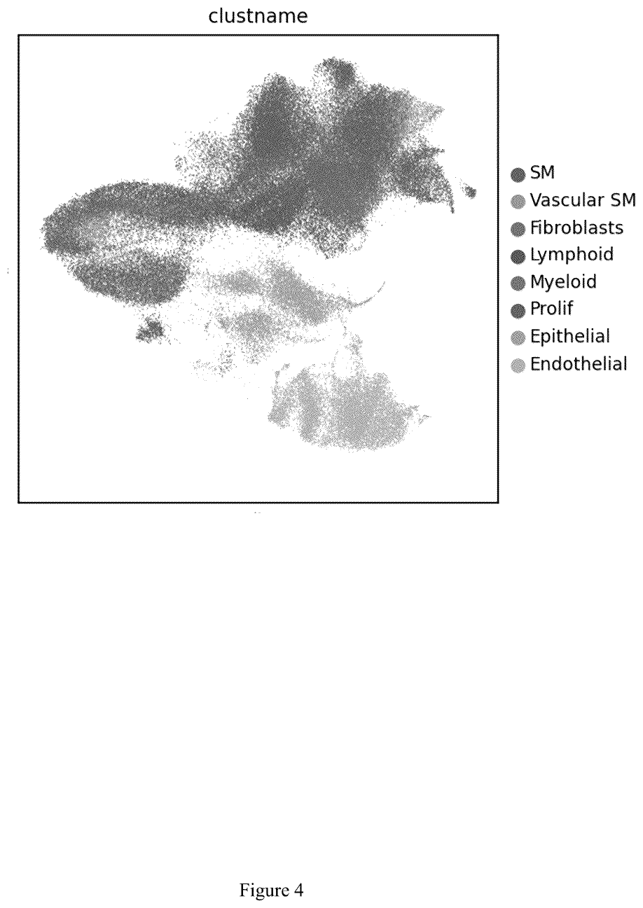

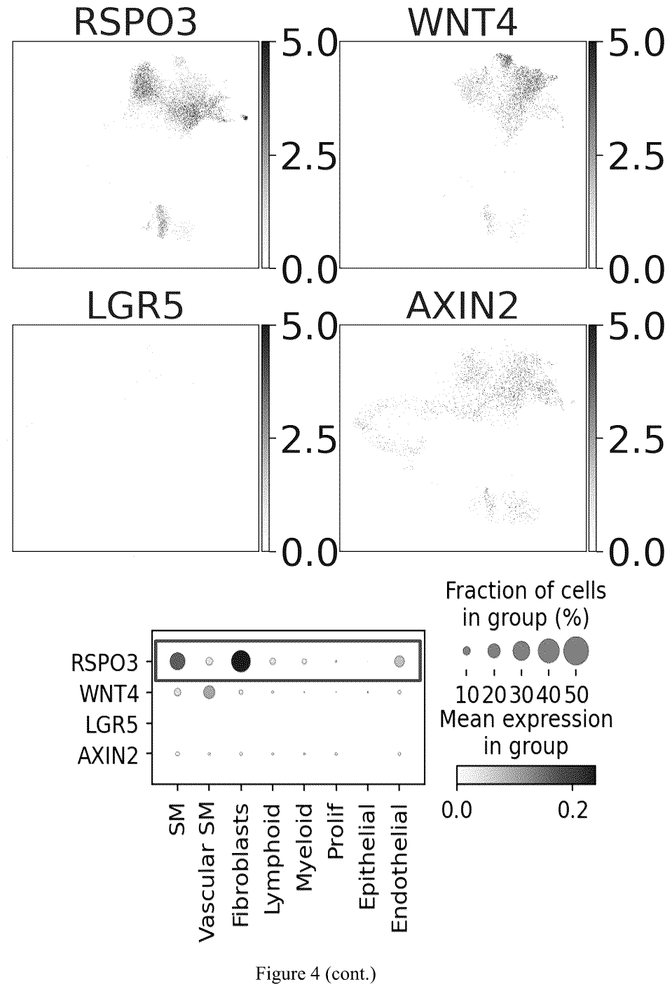

RSPO3 expression was observed mostly on smooth muscles and fibroblasts and some expression on endothelial cells (FIG. 4).

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having endometriosis, or at risk of developing endometriosis, the method comprising administering an R-Spondin (RSPO3) inhibitor and/or an RSPO3 Receptor blocker to the subject.

2. The method of claim 1, wherein the endometriosis comprises superficial peritoneal endometriosis, endometrioma-containing endometriosis, deeply infiltrating endometriosis (DIE), or abdominal wall endometriosis.

3. The method of claim 1, wherein the RSPO3 inhibitor comprises an inhibitory nucleic acid molecule that hybridizes to an RSPO3 nucleic acid molecule.

4. The method of claim 3, wherein the inhibitory nucleic acid molecule comprises an antisense nucleic acid molecule, a small interfering RNA (siRNA), and/or a short hairpin RNA (shRNA).

5. The method of claim 4, wherein the inhibitory nucleic acid molecule comprises an siRNA.

6. The method of claim 4, wherein the inhibitory nucleic acid molecule comprises an antisense nucleic acid molecule.

7. The method of claim 1, wherein the RSPO3 inhibitor comprises an antibody that decreases expression of the RSPO3 protein.

8. The method of claim 1, wherein the subject is also administered an endometriosis therapeutic agent or an endometriosis therapy.

9-13. (canceled)

14. The method of claim 13, further comprising detecting the presence or absence of an RSPO3 variant nucleic acid molecule in a biological sample from the subject.

15. The method of claim 14, further comprising administering an endometriosis therapeutic agent in a standard dosage amount or an endometriosis therapy to the subject when the RSPO3 variant nucleic acid molecule is absent from the biological sample.

16. The method of claim 14, further comprising administering an endometriosis therapeutic agent in a dosage amount that is the same as or less than a standard dosage amount or an endometriosis therapy to the subject when the subject is heterozygous or homozygous for the RSPO3 variant nucleic acid molecule.

17. The method of claim 14, wherein the RSPO3 variant nucleic acid molecule comprises a splice-site variant, a stop-gain variant, a start-loss variant, a stop-loss variant, a frameshift variant, a missense variant, an in-frame indel variant, and/or a variant that encodes a truncated RSPO3 variant polypeptide.

18. The method of claim 14, wherein the RSPO3 variant nucleic acid molecule comprises any one or more of the genetic variations 6:127125465:G:C (r59482771), 6:126727085:C:T (r5111743285), 6:126973497:T:C (r59398828), 6:127017612:T:C (r5117698897), 6:127114919:C:T (r51936800), 6:127118748:C:G (rs138650988), 6:127118902:T:C (rs577721086), 1:19979653:G:C (rs11573156), 3:56815721:T:C (rs1354034), 3:136318144:T:C (rs664088), 6:2507158:T:C (rs879097), 6:117184879:T:G (rs200564310), 6:154441670:T:C (rs75045626), 6:160671406:C:T (rs11751347), 6:160706469:A:G (rs73015965), 6:160716958:G:A (rs783147), 6:160731873:C:T (rs4252129), 6:160741337:T:A (rs147175166), 7:151716108:T:C (rs10224210), 8:105578477:C:A (rs4541868), 12:71474678:T:C (rs60195346), 13:73551661:G:A (rs1887646), 14:54766806:G:A (rs7147275), 14:54768564:C:T (rs4901541), 14:94371805:G:T (rs112635299), 16:251942:A:G (rs9934955), 16:3697041:G:T (rs1635404), 17:28367840:G:A (rs704), 19:44846145:T:C (rs3810143), 19:55027612:T:C (rs1654425), 20:45347027:A:C (rs1981430), 20:45354752:G:A (rs6065808), and 23:70475947:G:T.

19-20. (canceled)

21. A method of treating a subject having endometriosis or at risk of developing endometriosis by administering an endometriosis therapeutic agent or an endometriosis therapy, the method comprising:
determining or having determined whether the subject has an R-Spondin 3 (RSPO3) variant nucleic acid molecule, by:
obtaining or having obtained a biological sample from the subject; and
performing or having performed a sequence analysis on the biological sample to determine if the subject has a genotype comprising an RSPO3 variant nucleic acid molecule; and
administering or continuing to administer the endometriosis therapeutic agent or endometriosis therapy in a standard dosage amount to a subject that is RSPO3 reference; or
administering or continuing to administer the endometriosis therapeutic agent in an amount that is the same as or less than a standard dosage amount or endometriosis therapy to a subject that is heterozygous or homozygous for the RSPO3 variant nucleic acid molecule, and/or administering an RSPO3 inhibitor and/or an RSPO3 Receptor blocker to the subject;
wherein the presence of a genotype having the RSPO3 variant nucleic acid molecule indicates the subject has an increased risk of developing endometriosis.

22. The method of claim 21, wherein the RSPO3 inhibitor comprises an inhibitory nucleic acid molecule that hybridizes to an RSPO3 nucleic acid molecule.

23. The method of claim 22, wherein the inhibitory nucleic acid molecule comprises an antisense nucleic acid molecule, a small interfering RNA (siRNA), and/or a short hairpin RNA (shRNA).

24. The method of claim 23, wherein the inhibitory nucleic acid molecule comprises an siRNA.

25. The method of claim 23, wherein the inhibitory nucleic acid molecule comprises an antisense nucleic acid molecule.

26. The method of claim 21, wherein the RSPO3 inhibitor comprises an antibody that decreases expression of the RSPO3 protein.

27-31. (canceled)

32. The method of claim 21, wherein the subject is heterozygous or homozygous for the RSPO3 variant nucleic acid molecule, and the subject is administered or continued to be administered the endometriosis therapeutic agent in an amount that is the same as or less than a standard dosage amount or endometriosis therapy, and is administered the RSPO3 inhibitor and/or an RSPO3 Receptor blocker.

33. The method of claim 21, wherein the RSPO3 variant nucleic acid molecule comprises a splice-site variant, a stop-gain variant, a start-loss variant, a stop-loss variant, a frameshift variant, a missense variant, an in-frame indel variant, and/or a variant that encodes a truncated RSPO3 variant polypeptide.

34. The method of claim 21, wherein the RSPO3 variant nucleic acid molecule comprises any one or more of the genetic variations 6:127125465:G:C (r59482771), 6:126727085:C:T (r5111743285), 6:126973497:T:C (r59398828), 6:127017612:T:C (r5117698897), 6:127114919:C:T (r51936800), 6:127118748:C:G (rs138650988), 6:127118902:T:C (rs577721086), 1:19979653:G:C (r511573156), 3:56815721:T:C (rs1354034), 3:136318144:T:C (rs664088), 6:2507158:T:C (r5879097), 6:117184879:T:G (rs200564310), 6:154441670:T:C (r575045626), 6:160671406:C:T (rs11751347), 6:160706469:A:G (rs73015965), 6:160716958:G:A (rs783147), 6:160731873:C:T (r54252129), 6:160741337:T:A (r5147175166), 7:151716108:T:C (r510224210), 8:105578477:C:A (rs4541868), 12:71474678:T:C (r560195346), 13:73551661:G:A (r51887646), 14:54766806:G:A (r57147275), 14:54768564:C:T (r54901541), 14:94371805:G:T (rs112635299), 16:251942:A:G (rs9934955), 16:3697041:G:T (rs1635404), 17:28367840:G:A (r5704), 19:44846145:T:C (r53810143), 19:55027612:T:C (r51654425), 20:45347027:A:C (r51981430), 20:45354752:G:A (rs6065808), and 23:70475947:G:T.

35-68. (canceled)
